(19) United States
(12) Patent Application Publication
(10) Pub. No.: US 2025/0276169 A1
(43) Pub. Date: Sep. 4, 2025

(54) CIRCULATORY SUPPORT DEVICES, SYSTEMS, AND METHODS

(71) Applicant: Boston Scientific Scimed, Inc., Maple Grove, MN (US)

(72) Inventors: Judith Tiferes Wang, Shoreview, MN (US); Jennifer Gibbs, White Bear Township, MN (US)

(73) Assignee: Boston Scientific Scimed, Inc., Maple Grove, MN (US)

(21) Appl. No.: 19/065,523

(22) Filed: Feb. 27, 2025

Related U.S. Application Data
(60) Provisional application No. 63/559,355, filed on Feb. 29, 2024.

(51) Int. Cl. A61M 60/585; A61M 60/178

(52) U.S. Cl. CPC A61M 60/585; A61M 60/178; A61M 2205/10; A61M 2205/18; A61M 2205/3303; A61M 2205/3327; A61M 2205/3334; A61M 2205/3365; A61M 2205/502; A61M 2205/581; A61M 2205/584; A61M 2230/30

(57) ABSTRACT

A circulatory support system may include a blood pump, a console in communication with the blood pump, a first user interface with a first display, and a second user interface with a second display. The first display may be configured to present a series or array of functional status indicators relating to the operation of the blood pump. The second display may be configured to present real-time measured and/or estimated data and parameters related to operation of the blood pump. In an event where the second user interface and/or second display becomes inoperable, the first user interface and the first user display remain operational and continue displaying functional status indicators relating to operation of the blood pump.

### CROSS REFERENCE TO RELATED APPLICATIONS

The present application claims the benefit of U.S. Provisional Patent Application Ser. No. 63/559,355, filed Feb. 29, 2024, the disclosure of which is incorporated herein by reference.

### TECHNICAL FIELD

The present disclosure pertains to mechanical circulatory support devices, systems, and methods. More specifically, the present disclosure relates to percutaneous ventricular assist device (PVAD) systems with user interfaces and displays.

### BACKGROUND

A wide variety of intracorporeal and extracorporeal medical devices and systems have been developed for medical use, for example, in cardiac procedures and/or for cardiac treatments. Some of these devices and systems include guidewires, catheters, catheter systems, pump devices, circulatory assist devices, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing, display, operation and optimization of medical devices and systems.

### BRIEF SUMMARY

This disclosure provides design, material, method, and use alternatives for medical devices, including ventricular assist devices.

In a first example, a circulatory support system may comprise a blood pump, a console in communication with the blood pump, the console comprising a first user interface with a first display configured to present functional status indicators related to operation of the blood pump. The console further includes a second user interface with a second display that is configured to present values derived from measured and/or estimated real-time data obtained from the blood pump. In an event where the second interface and/or second display malfunctions or fails to operate, the first user interface and the first display remain operational with the first display continuing to present functional status indicators relating to the operation of the blood pump.

Additionally or alternatively, the first user interface and the first display may operate on a first operating system. Further, the second user interface and the second display may operate on a second operating system. The second operating system being a different operating system than the first operating system. Third, fourth, fifth, and myriad operating systems are contemplated as compatible with the present disclosure in all combinations and permutations of use and incorporation with the first user interface, first display, second user interface and second display.

Alternatively or additionally to any of the examples above, in another example, the first screen of the first user interface may be configured to display/present various patient parameters and blood pump parameters in addition to functional status indicators relating to operation of the blood pump of a circulatory support device such as a ventricular assist device or any other equivalent device.

Alternatively or additionally to any of the examples above, in another example, the second screen of the second user interface may be configured to display/present various real-time patient parameters based upon the measured/estimated data obtained from the blood pump of a circulatory support device such as a ventricular assist device or any other equivalent device.

Alternatively or additionally to any of the examples above, in another example, the console of the device may include an alarm connected to the first user interface, the first screen of the first user interface, and a backlight positioned on the console opposite the first user interface. Myriad backlight positions (i.e., back of the console, top of the console, side of the console, bottom of the console, etc.) elevations, inputs and outputs are contemplated.

The backlight of any of the embodiments of this disclosure may be connected to an alarm of the console and/or an alarm of the circulatory support system which is further connected to the first user interface and first screen of the console. As such, when an alarm condition is met, an alarm is sounded both on the console and displayed on a backlight of the console. The alarm sounds and alarm displays may correspond to differing alarm conditions based upon the severity of the alarm condition. A cautionary alarm condition, level-one, or first-level alarm condition may present a yellow light via the backlight while sounding a first sound or a grouping of sounds associated with a level-one alarm condition. A critical alarm condition, level-two or second-level alarm condition may present a red light via the backlight while sounding a second sound or a grouping of sounds both associated with a level-two alarm condition and different than the sounds announced by a level-one alarm condition.

Alternatively or additionally to any of the examples above, in another example, when the real-time estimated data obtained from the blood pump of a circulatory support device fails to meet a predetermined threshold, the second screen of the second user interface may present the real-time estimated data or real-time patient parameters in dashed-line, dotted-line or cross-hatched format. For example, if the blood flow rate real-time data is estimated below a preset threshold (e.g., less than 1.1 liter/min) then the presented value (e.g., 1.0 l/min) of the blood flow rate is shown in cross-hatched font on the second display of the second user interface.

Any and all real-time patient parameters and/or real-time estimated data may be shown in dashed-line, dotted-line or cross-hatched format when these values fail to meet a predetermined threshold programmed into the console of the circulatory support system. For further example, a threshold for Mean Arterial Pressure may be set at 50 mmHg, so that when the real-time estimated data of the Mean Arterial Pressure obtained from the blood pump of the circulatory support device falls beneath 50 mmHg, the Mean Arterial Pressure value is displayed in a dashed-line, dotted-line or cross-hatched font. It should also be noted that predetermined thresholds can include minimum thresholds, maximum thresholds, or outlier thresholds. An outlier threshold comprises a value that does not typically correspond to a set of given conditions. For example, if the estimated real-time data obtained from the pump shows blood flow rate values that do not typically correlate with obtained Mean Arterial Pressure values, one or both of the blood flow rate and Mean Arterial Pressure values may be represented by dotted-line, dashed-line or cross-hatched font.

Alternatively or additionally to any of the examples above, in another example, the second display of the second user interface may also present waveforms corresponding to the data obtained from the blood pump of a circulatory support device. Waveforms can be constructed for real-time data obtained from the blood pump, either measured or estimated. If the real-time measured and/or estimated data obtained from the blood pump fails to meet a predetermined threshold value, the waveform representing that data is presented in dashed-line, dotted-line or cross-hatched format.

Alternatively or additionally to any of the examples above, in another example, the second display of the second user interface may present data in two modes: Measured Mode and Estimation Mode. In Measured Mode, the data obtained from the blood pump of the circulatory support system is accurately measured directly from the blood pump. In Measured Mode, when accurate measurements are obtained, they are presented by the second display of the second user interface in solid-line fonts and waveforms. In Estimation Mode, the data obtained from the blood pump of the circulatory support system requires estimation and calculation by the console to present estimated values on the second display of the second user interface. The circulatory support system enters Estimation Mode due to myriad factors, including but not limited to: low battery status, minor pump malfunction, minor device malfunction, flow occlusion, operating system error, operating system malfunction, operating system crash, device occlusion, pump occlusion, poor internet connectivity, poor device connection, poor device position, poor pump connection, poor pump position, or other factors that could affect the accuracy of data obtained from the blood pump of the circulatory support system or device. When in Estimation Mode, where the data obtained must be calculated for estimation, the estimated values presented on the second display of the second user interface are presented in dashed-line, dotted-line or cross-hatched font and format.

Circulatory support systems contemplated by this disclosure include any device capable of circulatory support that is compatible with a user interface. Example circulatory support systems include but are not limited to left ventricular assist devices (LVAD), right ventricular assist devices (RVAD), biventricular assist devices (BiVAD), intraaortic balloon pumps (IABP), heart pumps, impeller pumps, artificial hearts, total artificial hearts (TAH) or any of the like or equivalent.

The data obtained from the blood pump of a circulatory support device and relayed to the second user interface may be updated and reflected on the second screen in synchronization with the heartbeat of the user of the blood pump (i.e., patient), e.g., with every heartbeat of the user of the blood pump (i.e., patient).

Alternatively or additionally to any of the examples above, in another example, the first user interface may include a first display having a first surface area and the second user interface may include a second display having a second surface area greater than the first surface area.

Alternatively or additionally to any of the examples above, in another example, the first user interface may be configured to receive one or both of functional status indicator data and real-time measured and/or estimated data obtained from the blood pump over a local area network or a wide area network. Further, the first display may be configured to present one or both of functional status indicator data and real-time measured and/or estimated data obtained from the blood pump.

Alternatively or additionally to any of the examples above, in another example, the second user interface may be configured to receive one or both of functional status indicator data and real-time measured and/or estimated data obtained from the blood pump over a local area network or a wide area network. Further, the second display may be configured to present one or both of functional status indicator data and real-time measured and/or estimated data obtained from the blood pump.

Alternatively or additionally to any of the examples above, in another example, the console may be configured to communicate with the blood pump over a wired connection.

Alternatively or additionally to any of the examples above, in another example, the console may be configured to communicate with the blood pump over a wireless connection.

In another example, a method is disclosed of operating a circulatory support system. The method includes the steps of utilizing at least one user display integrated with a console of a circulatory support system to display functional status indicator values and real-time measured and/or estimated data obtained from a blood pump of a circulatory support device. The method further incorporates the step of receiving data on at least a first display of at least one user interface, whereby if the device is in a first-level alarm condition (i.e., cautionary alarm condition) or a second-level alarm condition (i.e., critical alarm condition), the at least first display generates and presents instructions regarding troubleshooting the circulatory support device.

Alternatively or additionally to any of the examples above, in another example, the method may further comprise receiving an alarm alert corresponding to one or both of a first-level alarm condition and a second-level alarm condition via a backlight positioned on the console of a circulatory support system. The backlight may alert the user with a yellow light for a first-level alarm condition (i.e., cautionary alarm condition), and may alert the user with a red light for a second-level alarm condition (i.e., critical alarm condition). Other color-codes, alerts and alarm conditions are contemplated by the methods, devices and systems disclosed. Further, the backlight visual alarm may occur simultaneously with a visual alarm beaming on the at least first display and with an audio alarm sounding from the console of the circulatory support system simultaneously. The backlight may be positioned anywhere on the console, but in certain embodiments, the backlight is positioned on the back of the console whereby the at least first user interface and first screen are positioned on the front of the console.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a schematic diagram of an illustrative circulatory support system with a blood pump positioned in a patient.

FIG. 2 is a flow chart depicting the connectivity of structural elements regarding a circulatory support system with a blood pump connected to a first user interface and a second user interface.

FIG. 3 shows a front view of an exemplary console of the present disclosure.

FIG. 4 shows a back view of an exemplary console of the present disclosure.

FIG. 5 is a depiction of a first display and a second display both in normal operating condition as presented on an exemplary console of the present disclosure.

FIG. 6 shows a depiction of tactile speed buttons and speed button interfaces of the console in embodiments of the present disclosure.

FIG. 7A is a representation of relatively blank first and second displays on the console in an embodiment of the present disclosure.

FIG. 7B is a representation of all functional status indicators displayed on a first display of the console in an embodiment of the present disclosure.

FIG. 7C is a representation of a pump connection malfunction as shown on the first and second displays of the console in an embodiment of the present disclosure.

FIG. 7D is a representation of a working pump connection as shown on the first and second displays of the console in an embodiment of the present disclosure.

FIG. 7E is a representation of the first and second displays of the console in an embodiment where the console is in full working order.

FIG. 7F is a representation of the first and second displays of the console in a scenario where the displayed blood flow rate through the blood pump has a low confidence of accuracy and/or its accuracy is uncertain, and is thus displayed in cross-hatched font according to another embodiment of the present disclosure.

FIG. 7G is a representation of the first and second displays of the console in an embodiment in which waveforms of the Mean Arterial Pressure (MAP) and the flow rate through the blood pump are displayed.

FIG. 8 is a flowchart depicting the instructions generated by one or more of the first and second displays regarding troubleshooting of the circulatory support system of the present disclosure.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a” “an”, and “the” include plural referents unless the content clearly dictates otherwise. As used in this specification and the appended claims, the term “or” is generally employed in its sense including “and/or” unless the content clearly dictates otherwise.

Unless stated to the contrary, the terms “display” and “screen” are considered synonymous for the purposes of this disclosure. For instance, first display and first screen depict the same element of the disclosure unless otherwise indicated.

It is noted that references in the specification to “a configuration”, “another configuration”, “some configurations”, “other configurations”, etc., indicate that the configuration described may include one or more particular features, structures, and/or characteristics. However, such recitations do not necessarily mean that all embodiments include the particular features, structures, and/or characteristics. Additionally, when particular features, structures, and/or characteristics are described in connection with one configuration, it should be understood that such features, structures, and/or characteristics may also be used in connection with other configurations whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure. Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

A variety of circulatory support devices and systems are known for assisting or replacing a pumping function of a heart in a patient with severe heart failure and/or other cardiac conditions. Circulatory support devices may be configured to treat patients with cardiogenic shock, myocardial infarction, acutely decompensated heart failure, and/or other heart related conditions. Additionally or alternatively circulatory support devices may support a patient during percutaneous coronary interventions and/or other procedures.

Example cardiac circulatory support devices include, but are not limited to, ventricular assist devices (VADs), total artificial hearts (TAH), intra-aortic balloon pumps (IABP), and extracorporeal membrane oxygenation (ECMO). Example VADs include left ventricular assist devices (LVADs), right ventricular assist devices (RVADs), and biventricular assist devices (BiVADs). A further illustrative VAD is a percutaneous ventricular assist device (PVAD), which may be inserted into a ventricle (e.g., a left ventricle or a right ventricle) of a heart of a patient via delivery through a femoral artery or vein and/or other suitable vasculature to the ventricle. As a PVAD may be placed at a desired location of anatomy of a patient via percutaneous access and delivery, the PVAD may be used in emergency medicine, a catheter laboratory, and/or other surgical and/or non-surgical settings.

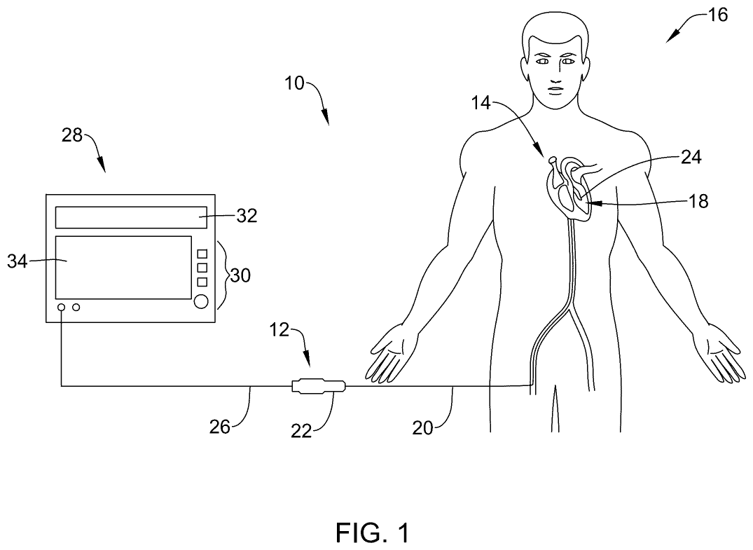

FIG. 1 depicts an illustrative percutaneous circulatory support system 10 including a circulatory support device 12 positioned in the heart 14 of a patient 16. The circulatory support device 12 may include, among other components, a flexible elongate catheter shaft 20 having a first end attached to a housing 22 and a second end attached to a blood pump 24. As depicted in FIG. 1, the blood pump 24 may be positioned in the left ventricle 18 of the patient 16. The blood pump 24 may be delivered (e.g., tracked) to the left ventricle 18 percutaneously over a guidewire. For example, the catheter shaft 20 and the blood pump 24 may be tracked over a guidewire through the femoral artery and the descending aorta, over the aortic arch, through the ascending aorta, past the aortic valve, and into the left ventricle 18.

In some examples, the blood pump 24 may be positioned within the heart 14 such that one or more blood inlets positioned along the distal region of the blood pump 24 may be located in the left ventricle 18 and one or more blood outlets positioned along a housing of the blood pump 24 may be located in the aorta. Additionally, the blood pump 24 may include an electrically powered motor that drives rotation of an impeller (e.g., where the motor and/or the impeller may be positioned within the housing of the blood pump). In some examples, the motor may power the rotation of the impeller via electromagnetic induction, but other suitable configurations are contemplated. The rotating impeller may draw blood from the left ventricle (via the one or more of the blood inlets) into the aorta (via the one or more blood outlets). In other words, an electrically powered motor may drive the impeller to pump blood from the left ventricle through the aortic valve and into the ascending aorta.

FIG. 1 further illustrates that a first end of the catheter shaft 20 may be attached to a housing 22. The housing 22 may include a distal end region attached to the catheter shaft 20 and a proximal end region attached to a cable 26. It can be appreciated that the housing 22 may include one or more actuators (e.g., buttons, levers, dials, switches, etc.) designed to permit a clinician to control various functions of the blood pump 24. For example, a clinician may be able to control the speed of the motor and/or the impeller located in the blood pump 24 via actuation of one or more actuators located on the housing 22 in some instances.

The cable 26 may include a proximal end region connected to a console 28. The cable 26 may include or be an electrical power cable configured to power the blood pump 24, a communication cable configured to communicate data between the blood pump 24 and the console 28, an electrical cable, an optical cable, and/or other suitable type of cable.

The circulatory support system 10 may include one or more sensors positioned within and/or along the console 28, the housing 22, the catheter shaft 20, and/or the blood pump 24, where the sensors may be coupled with the console 28 and/or other data monitoring component via wired connection (e.g., an electrical and/or optical connection of the cable 26) and/or a wireless connection. The one or more sensors positioned within and/or along the console 28, within the housing 22, the catheter shaft 20, and/or the blood pump 24 may be configured to sense one or more parameters of or related to operation of the circulatory support system 10, operation of the circulatory support device 12, operation of the blood pump 24 and/or the patient. The one or more sensors positioned within and/or along the console 28, the housing 22, along the catheter shaft 20, and/or the blood pump 24 may be designed or configured to sense and/or monitor any suitable parameters including, but not limited to, blood pressures (e.g., arterial pressure, venous pressure), blood velocity, blood flow rate, blood flow rate through the blood pump, mean blood flow rate, mean blood flow rate through the blood pump, minimum blood flow rate, minimum blood flow rate through the blood pump, maximum blood flow rate, maximum blood flow rate through the blood pump, mean arterial pressure, minimum arterial pressure, maximum arterial pressure, impeller speeds, motor speeds, an electrical current provided to the motor, voltage provided to the motor, back-EMF from the motor, and/or other suitable parameters, along with any suitable combination or temporal pattern of signals corresponding to one or more of the parameters. Further, additional parameters (e.g., flow through or across the blood pump 24) may be derived by processing combinations of sensed data in a time dependent manner.

As depicted in FIG. 1, the console 28 is connected to a blood pump 24 of a circulatory support system 10 and may include a first user interface 32 and a second user interface 34. FIG. 1 depicts the console 28 with a first user interface 32 comprising a first display and a second user interface 34 comprising a second display, but other suitable configurations of the console 28 with a single display and/or with more than two displays are contemplated. Additionally, while FIG. 1 illustrates the first display and the second display integrated into the console 28, it is contemplated that the circulatory support system 10 may be designed such that the first display, the second display, or both of the first display and the second display are separate, distinct components of the circulatory support system 10. In other words, the first display, the second display, or both of the first display and the second display may be separate stand-alone displays, apart from the console 28, and/or the first display or user interface 32 may be separate from the second display or user interface 34 and contained in separate housings. In some examples, the first display and the second display may receive data from the same or different sources. An example circulatory support system including a console with two displays is disclosed in Appl. No. 63/457,935, filed on Apr. 7, 2023, and titled CIRCULATORY SUPPORT DEVICE SYSTEM, which is hereby incorporated by reference in its entirety for any and all purposes.

The first display of the first user interface 32 of the console 28 may be configured to display functional status indicators related to the operation of a blood pump 24 of a circulatory support system 10.

The second display of the second user interface 34 of the console 28 may be configured to display real-time patient parameter values based on measured and/or estimated real-time data obtained from a blood pump 24 of a circulatory support system 10. Additionally, the second display may also be configured to present functional status indicators relating to the operation of a blood pump 24 of a circulatory support system 10. Such functional status indicators may include, but are not limited to pump connectivity status, device connectivity status, motor rotation status, pump power status, device power status, circulatory support system power status, circulatory support system connectivity status, pump occlusion status, pump suction status, pump position status, device position status, battery level status, low battery level status, critically low battery level status, or any equivalent or suitable functional status indicator.

The computing device or controller that operates the console 24 may be any suitable computing device, controller or operating system configured to process data of or for the circulatory support system 10 and may be configured to facilitate operation of the circulatory support system 10. The computing device, operating system or controller, in some cases, may be configured to control operation of the blood pump 24 by establishing and/or outputting control signals to the blood pump 24 and/or components of or in communication with the console 28 to control and/or monitor operation of the blood pump 24. In some examples, the controller may be part of the console 28 and communicate with the blood pump 24 over a wired connection, but other suitable configurations are contemplated.

The first user interface 32 and first display of the console 28 may run on a first operating system. Further, the second user interface 34 and second display of the console 28 may run on a second operating system. The first operating system being a different operating system than the first operating system. Myriad operating systems are contemplated for compatibility with the first user interface 32, second user interface 34, first display, second display and console 28 of the present disclosure including all possible permutations and combinations of their utilization. Contemplated operating systems include, but are not limited to: iOS, Mac OS, Linux, Windows, Fire OS, DOS, Apple DOS, Unix, CP/M, FlexOS, Android, HDOS, iRMX, iMAX 432, FORTRAN, DOS/360 or any other equivalent or suitable operating system.

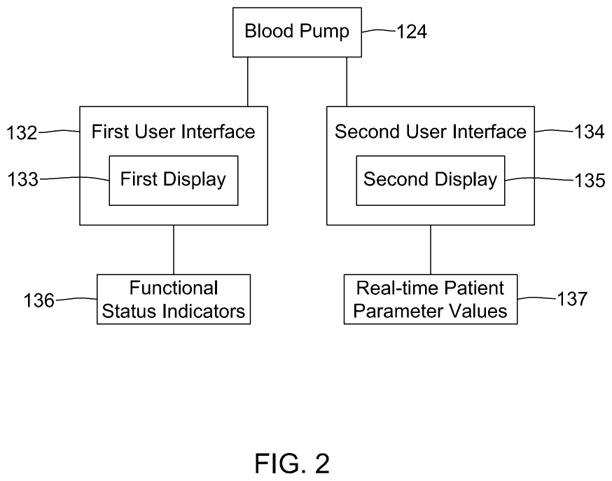

FIG. 2 illustrates a flow chart showing the connectivity of elements of the system of the present disclosure. As shown, a blood pump 124 (which may be the blood pump 24 of FIG. 1) from a circulatory support system, such as the circulatory support system 10 of FIG. 1, is connected to a first user interface 132 and a second user interface 134. The first user interface 132 may represent the first user interface 32 of FIG. 1 and/or the second user interface 134 may represent the second user interface 34 of FIG. 1. The first user interface comprises a first display 133, while the second user interface comprises a second display 135. The second display 135 may be separate from and spaced apart from the first display 133, and may be controlled by a separate controller or operating system than the first display 133. The flow chart further illustrates that the first display 133 shows functional status indicators 136 relating to operation of the blood pump 124 of the circulatory support system. The flow chart also illustrates that the second display 135 shows real-time patient parameter values 137 derived from measured and/or estimated data obtained from the blood pump 124 of the circulatory support system.

Exemplary real-time patient parameter values include but are not limited to: minimum arterial pressure, maximum arterial pressure, mean arterial pressure, minimum blood flow rate, maximum blood flow rate, mean blood flow rate, minimum blood flow rate through the blood pump, maximum blood flow rate through the blood pump, mean blood flow rate through the blood pump, estimated blood pressure, estimated blood pressure waveform, left ventricular pressure, minimum left ventricular pressure, maximum left ventricular pressure, mean left ventricular pressure, minimum arterial oxygenation, maximum arterial oxygenation, mean arterial oxygenation, minimum arterial pressure waveform, continuous arterial pressure waveform, maximum arterial pressure waveform, mean arterial pressure waveform, blood flow rate waveform, continuous blood flow rate waveform, minimum blood flow rate waveform, maximum blood flow rate waveform and mean blood flow rate waveform. Additionally, the real-time patient parameter values are obtained from the blood pump 124 (e.g., one or more sensors incorporated with the blood pump 124) of the circulatory support system or other sensors usable in association with the circulatory support system in real time as the blood pump 124 is in operation in the patient's heart. For example, in some instances the real-time patient parameter values may be obtained from one or more sensors and updated on the first display 133 and/or second display 135 in synchronization with the heartbeat of the patient, e.g., with every patient heartbeat. In other words, real-time data may be obtained and calculated into values that are updated by the console 28 and presented on one or both of the first display 133 and second display 135 with each and every patient heartbeat.

Exemplary functional status indicators include but are not limited to: low battery status, critically low battery status, ventricular assist device connection, ventricular assist device position, pump connection, pump position, motor status, motor rotation status, motor speed status, flow rate status, restricted flow rate status, flow occlusion status, ventricular assist device occlusion status, pump occlusion status, calibration status, recalibration status, Mean Arterial Pressure (MAP) status, alarm status, lock status, unlock status, backlight status, pump speed status, pump operational status and ventricular assist device status. Additionally, the functional status indicators may be updated by the console 28 in real-time, e.g., with every patient heartbeat. In other words, functional status indicators are obtained and calculated and their updates are presented on one or both of the first display 133 and second display 135 throughout operation of the blood pump 24, e.g., with each and every patient heartbeat.

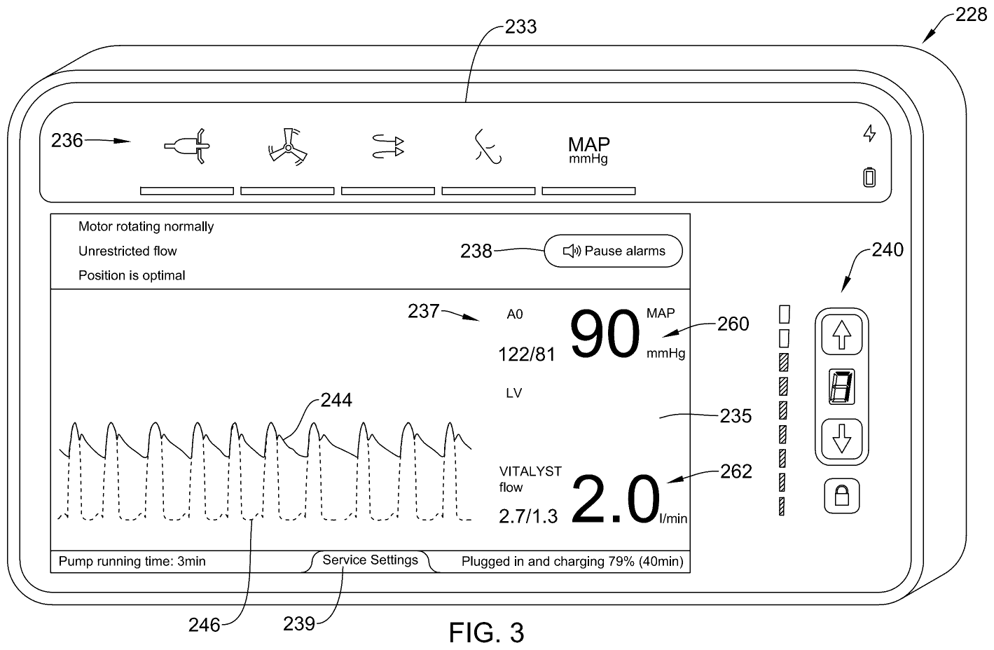

FIG. 3 shows an embodiment of a console 228 of the present disclosure. The console 228 may be representative of the console 28 of FIG. 1. The console 228 may include s first display 233, which may be representative of the first user interface 32 of FIG. 1, and a second display 235, which may be representative of the second user interface 34 of FIG. 1. As shown, the first display 233 is situated at the top of the front of the console 228. The first display 233 presents an array of functional status indicators 236, which from left-to-right correspond to circulatory support system connectivity (e.g., whether the circulatory support system is electrically connected properly to a power source), motor rotation status (e.g., whether the motor powering the blood pump is running), blood pump suction/occlusion status (e.g., whether there is an obstruction of blood flow), circulatory support system/blood pump positioning status (e.g., whether the blood pump is correctly positioned across the aortic valve with the blood inflow on the ventricular side of the aortic valve and the blood outflow on the aortic side of the aortic valve) and Mean Arterial Pressure status (e.g., whether the Mean Arterial Pressure is within an operational/acceptable range) as retrieved from a blood pump of a circulatory support system.

FIG. 3 further illustrates the second display 235 shown situated beneath or below the first display 233 of the console 228. The second display 235 in this embodiment presents real-time patient parameters derived from measured and/or estimated data obtained from the blood pump of the circulatory support system. The second display 235 may display one or more real-time parameters 237 (e.g., real-time patient and/or system parameters). For example, the second display 235 may display a real-time parameter 260 (i.e., value) of a measured and/or estimated blood pressure of the patient, such as Mean Arterial Pressure, minimum arterial pressure, maximum arterial pressure, left ventricular pressure, etc. The Mean Arterial Pressure (MAP) is shown in FIG. 3. Additional or alternatively, the second display 235 may display a real-time parameter 262 (i.e., value) of a measured and/or estimated blood flow rate, such as mean blood flow rate through the blood pump, minimum blood flow rate through the blood pump, maximum blood flow rate through the blood pump, etc. The mean blood flow rate through the blood pump is shown in FIG. 3. Additional real-time parameters may also be similarly displayed, such as the left ventricular pressure of the patient.

The second display 235 may also show a waveform associated with one or more of the real-time parameters 237 displayed on the second display 235. For instance, the second display 235 may include a waveform depicting live (i.e., real-time) patient and/or system parameters associated with one or more of the displayed real-time parameters 237. For instance, the second display 235 may show a first waveform 244 associated with the real-time parameter 260 (i.e., value) of a measured and/or estimated blood pressure of the patient, e.g., Mean Arterial Pressure. Additionally or alternatively, the second display 235 may show a second waveform 246 associated with another real-time parameter, such as a measured and/or estimated left ventricular pressure of the patient.

The second display 235 of FIG. 3 further shows functional status indicators in the top-left corner of the second display 235. In this embodiment, the functional status indicators regard motor function/status, blood flow rate through the blood pump/occlusion status, and device/blood pump positioning status, blood pump/device power status, and console battery level status. In some instances, these functional status indicators displayed in the second display 235 may correspond to one or more of the functional status indicators 236 shown on the first display 233.

Further represented in the second display of FIG. 3 are an embedded mute alarm button 238 on the second display 235 (top-right corner of second display 235), and a service settings pull-down/pull-up menu 239 (e.g., a tab) also accessible by a touch-screen interface of the second display 235 (shown at the center-bottom of the second display 235 in FIG. 3). Lastly, speed control tactile buttons 240 are shown on the right side of the console 228. The speed control tactile buttons 240 may include, but are not limited to speed up button for increasing the speed of the blood pump, speed down button for decreasing the speed of the blood pump, speed hold button, speed lock button, speed unlock button, device lock button, console lock button, or any equivalent input button known in the art. The speed control buttons 240 may be used to set and/or control the speed of the blood pump at one or more discrete speed levels, in some instances.

The illustrative console 228 may include, among other suitable components, one or more processors, operating systems, one or more memory, and/or one or more I/O units. Example other suitable components of the console 228 that are not specifically depicted in FIG. 3 may include, but are not limited to, communication components, a touch screen, selectable buttons, and/or other suitable components of a console.

The memory of the console 228 may include a single memory component or more than one memory component each working individually or with one another. Example types of memory may include random access memory (RAM), EEPROM, flash, suitable volatile storage devices, suitable non-volatile storage devices, persistent memory (e.g., read only memory (ROM), hard drive, flash memory, optical disc memory, and/or other suitable persistent memory) and/or other suitable types of memory. The memory may be or may include a non-transitory computer readable medium. The memory may include instructions stored in a transitory state and/or a non-transitory state on a computer readable medium that may be executable by the processor or operating system to cause the processor or operating system to perform one or more of the methods and/or techniques described herein. Further, in some cases, the memory and/or other suitable memory may store data received from the blood pump (e.g., one or more sensors incorporated with the blood pump) of the circulatory support system or other sensors usable in association with the circulatory support system in real time as the blood pump is in operation in the patient's heart.

The I/O units of the console 228 may include a single I/O component or more than one I/O component each working individually or with one another. Example I/O units may be or may include any suitable types of communication hardware and/or software including, but not limited to, communication components or ports configured to communicate with electronic components of the circulatory support system and/or with other suitable computing devices or systems. Example types of I/O units may include, but are not limited to, wired communication components (e.g., HDMI components, Ethernet components, VGA components, serial communication components, parallel communication components, component video ports, S-video components, composite audio/video components, DVI components, USB components, optical communication components, and/or other suitable wired communication components), wireless communication components (e.g., radio frequency (RF) components, Low-Energy BLUETOOTH protocol components, BLUETOOH protocol components, Near-Field Communication (NFC) protocol components, WI-FI protocol components, optical communication components, ZIGBEE protocol components, and/or other suitable wireless communication components), and/or other suitable I/O units.

The first user interface 233 may be configured to communicate with the computing device or controller via one or more wired or wireless connections. The first user interface 233 may include one or more displays, one or more input devices, one or more output devices, and/or one or more other suitable features. In some examples, the first user interface 233 may be part of or may include the computing device or controller.

The second user interface 235 may be configured to communicate with the computing device or controller via one or more wired or wireless connections. The second user interface 235 may include one or more displays, one or more input devices, one or more output devices, and/or one or more other suitable features. In some examples, the second user interface 235 may be part of or may include the computing device or controller.

The displays of the present disclosure may be any suitable display. Example suitable displays include, but are not limited to, touch screen displays, non-touch screen displays, gesture displays, haptic displays, tactile displays, liquid crystal display (LCD) screens, light emitting diode (LED) displays, head mounted displays, 4K pixel displays, 8K pixel displays, 16K pixel displays, 32K pixel displays, virtual reality displays, augmented reality displays, virtual reality displays and/or other suitable display types.

The input device(s) may be and/or may include any suitable components and/or features for receiving user input via the user interfaces of the present disclosure. Example input device(s) may include, but are not limited to, touch screens, keypads, mice, touch pads, microphones, selectable buttons, selectable knobs, optical inputs, cameras, gesture sensors, eye trackers, voice recognition controls (e.g., microphones coupled to appropriate natural language processing components) and/or other suitable input devices. In one example, the input devices may include a touch screen that allows for setting set points and/or selecting selectable elements for additional detail concerning data or information associated with the selectable elements, but this is not required.

The console 228 of the circulatory support system may be configured to facilitate control of the blood pump and store and/or monitor data related to operation of the blood pump (e.g., data from the blood pump, data from control of the blood pump, data from sensors of and/or associated with the blood pump, and/or data from other suitable sources). The console 228 may receive data over time from the blood pump operating within a patient, where the received data may include patient data and/or data related to operation of the blood pump within the patient.

The console 228 may be configured so as to be sized and coupled to the blood pump in a manner that allows for transporting the console 228 with the patient in which the blood pump has been positioned, which may include transporting the patient via an emergency medical services (EMS) (e.g., via helicopter, ambulance, etc.) and/or transporting the patient between locations within a medical facility, such as a hospital, (e.g., between a catheter laboratory, an operating room, an intensive care unit, and/or other location within the medical facility). To assess the patient and/or operation of the blood pump, medical professionals may need to evaluate historical patient data and/or blood pump data gathered and/or saved at or by the console 228.

To facilitate transportation of the console 228 with the patient, the console 228 may be lightweight and/or compact. However, it may be desirable for the console 228 to have a large display configured to facilitate displaying patient data and/or blood pump data that facilitates medical professionals assessing the patient.

To address the competing design concerns (e.g., lightweight and/or compact versus a large display for obtaining a full understanding of a patient's condition and/or a pump condition), a console 228 may include a first display 233, which may be configured to display one or more screens providing limited data or information (e.g., preconfigured screens with fixed layouts), and a second display 235 which may include or may be coupled with a second user interface which may display values having augmented views relative to what is depicted on the first display 233.

The second display 235 may be incorporated into the same housing of the console 228 as the first display 233, yet the second display 235 may be controlled by a separate controller or operating system than the first display 233. Thus, if the operating system or controller operating the second display 235 fails, crashes, or otherwise becomes inoperable, the first display 233 may remain functional and operational such that operation of the blood pump of the circulatory support system may continue uninterrupted while still displaying operational status of the blood pump to the medical personnel to using the functional status indicators of the first display 233.

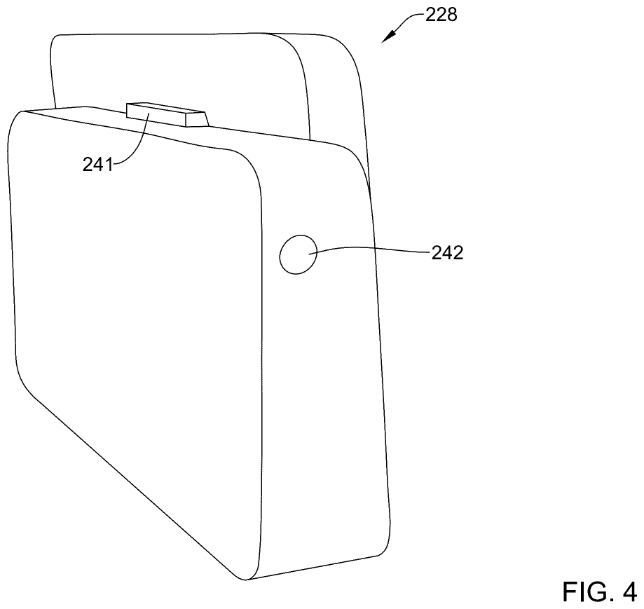

FIG. 4 displays a back-end view of an embodiment of the console 228 of the present disclosure. At the back of the console 228, atop a ridge or ledge behind the first display of the console 228 sits a backlight 241. The backlight 241 may be viewable by medical personnel standing behind the console 228, or otherwise visible from the rear side of the console 228. In other embodiments, the backlight 241 may be positioned at a different location visible from the rear side of the console 228. The backlight 241 is configured to alert a user/practitioner/patient of the device to an alarm condition relating to operation of the circulatory support system. For example, when an alarm notification is displayed on the first display 233 and/or the second display 235, the backlight 241 may also be correspondingly illuminated. Thus, medical personnel not in line-of-sight of the front of the console 228 (i.e., medical personnel not able to view the first display 233 and/or the second display 235) may be nonetheless notified of a system alarm/notification/status via the backlight 241, prompting the medical personnel to view the first display 233 and/or the second display 235 to determine the status of the blood pump and/or circulatory support system.

On a side of the console 228, sits an on/off button 242. The on/off button may be a depressible button, a tactile button, a touch button, a gesture button, an embedded button, a projecting button, a flat button, or any other input construction known in the art.

The backlight 241 of the console 228 may be constructed and configured in various illumination designs, formats, textures and modes. For example, the backlight 241 may be a singular LED or an array of LEDs. Myriad other designs, formats, textures and modes of illumination are contemplated as well as any equivalent illumination component(s) known in the art for incorporation into the backlight 241 of the console 228 of the present disclosure. Further, multiple backlights 241 may be present on one or more surfaces of the console 228.

The backlight 241 of the console 228 may also be configured to correspond with audio alarm alerts/notifications generated by the console 228 by simultaneously producing visible light (e.g., a flashing light) to indicate the proper alarm condition. For example, a first-level or cautionary alarm condition may be shown as a first color of light, such as yellow light (e.g., a yellow flashing light or a yellow solid light) on the backlight 241. Additionally, a second-level or critical alarm condition may be shown as a second color of light different from the first color of light, such as a red light (e.g., a red flashing light or a red solid light) on the backlight 241. In some instances, when an alarm notification is displayed on the first display 233 and/or the second display 235 in a first color of light, the backlight 241 may also be correspondingly illuminated in the same color of light. In instances, in which multiple alarm notifications are displayed on the first display 233 and/or the second display 235 with different colors of light, the backlight 241 may be correspondingly illuminated with both colors of light and/or with the light associated with the most serious or critical alarm notification. Myriad formats for displaying light are also contemplated, including but not limited to steady light, pulsating light, patterned light, strobing light, patterned intervals of flashing light, patterned intervals of pulsating light, irregular intervals of flashing light, irregular intervals of pulsating light or any other routine of illumination known in the art.

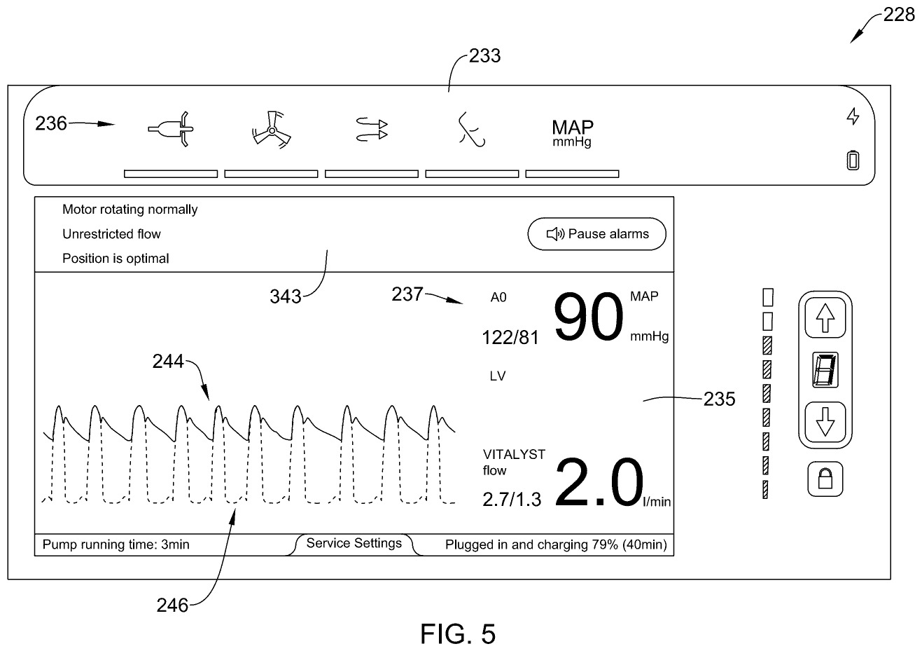

FIG. 5 illustrates a view of the console 228 of the present disclosure during conflict-free operation of the circulatory support system. The first display 233 of FIG. 5 displays functional status indicators 236 in a horizontal array. From left-to-right, the functional status indicators include blood pump/device connectivity status, motor rotation status, blood flow through the blood pump status, blood pump/device position status, Mean Arterial Pressure (MAP) status, blood pump/device power status, console battery level status.

The second display 235 of FIG. 5 illustrates several patient parameters 237 and functional status indicators 343. The top-left of the second display 235 presents functional status indicators 343 relating to the function of a blood pump of a circulatory support system in a vertical array with motor rotation status at the top, status of blood flow rate through the blood pump in the middle, and device/blood pump position status at the bottom of the vertical array. Additionally, the order and arrangement of functional status indicators 343 may be changed or altered to suit the purposes of the practitioner of the device. In some instances, these functional status indicator 343 displayed in the second display 235 may correspond to one or more of the functional status indicators 236 shown on the first display 233.

The second display 235 of FIG. 5 further presents patient parameters including a waveform 244 of Mean Arterial Pressure continuously measured or calculated over a period of time, drawn over a waveform 246 of left ventricular pressure continuously measured or calculated over a period of time. The second display 235 may also present other patient parameters, such as maximum arterial pressure, minimum arterial pressure, Mean Arterial Pressure, minimum blood flow rate through the blood pump, maximum blood flow rate through the blood pump and mean blood flow rate through the blood pump. Further shown on the second display 235 are an alarm control button in the top-right and a service settings pull-down/pull-up menu (e.g., tab) in the center-bottom of the second display 235. Additionally, the order and arrangement of patient parameters and selectable options may be changed or altered to suit the purposes of the practitioner of the device.

Waveforms can be constructed for real-time data obtained from the blood pump, either measured or estimated. The obtained data including but not limited to measured blood pressure data, estimated blood pressure data, measured arterial pressure data, estimated arterial pressure data, measured and/or estimated minimum arterial pressure data, measures and/or estimated maximum arterial pressure data, measured Mean Arterial Pressure data, estimated Mean Arterial Pressure data, measured blood flow rate through the blood pump data, estimated blood flow rate through the blood pump data, measured and/or estimated minimum blood flow rate through the blood pump data, measured and/or estimated maximum blood flow rate through the blood pump data or measured and/or estimated mean blood flow rate through the blood pump data.

In some instances, the waveforms may be depicted in different colors, line weight, and/or line style depending on the parameter displayed. For instance, when waveform data obtained from the blood pump of a circulatory support device meets a predetermined threshold for acceptable values, the waveform may be presented in a solid-line format. However, if the waveform data fails to reach a predetermined programmed threshold, then the waveform may be shown in dotted-line, dashed-line, or cross-hatched format.

In some instances, measured data may be displayed in a solid-line format waveform, whereas estimated data may be displayed in a dotted-line, dashed-line, or cross-hatched format waveform. In some instances, waveform data having a high confidence of accuracy may be displayed in a solid-line format waveform, whereas waveform data having a low confidence of accuracy and/or uncertain accuracy may be displayed in a dotted-line, dashed-line or cross-hatched format waveform.

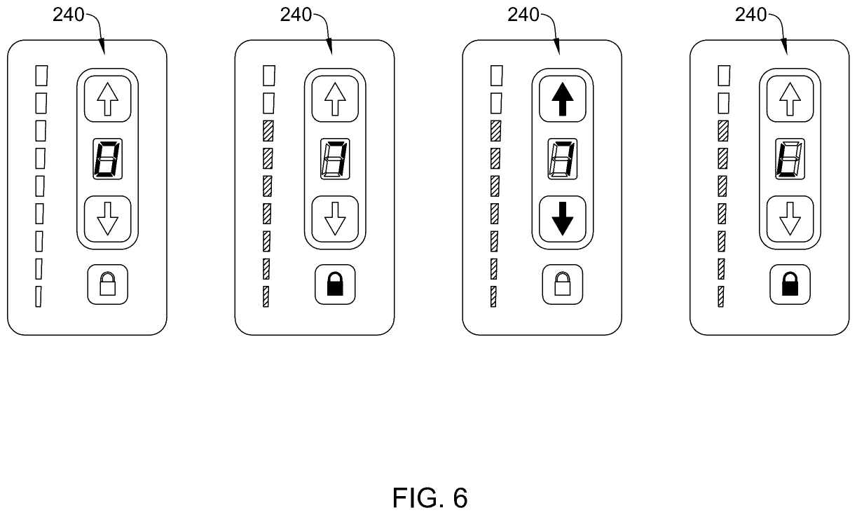

FIG. 6 is a close-up view of some various exemplary speed button scenarios of the speed control buttons 240 presented by speed button interfaces of the console 228. The speed buttons 240 may be tactile, haptic or comprise any other equivalent means of input known in the art. As shown in FIG. 6, the left-most speed button interface shows the blood pump operating at a speed of 0. If the user wishes to increase the speed of the pump, the user may depress the speed up button (e.g., button with the “up” arrow). As shown in the second speed button interface of FIG. 6, the user has set the pump speed to level 7 and has locked the speed button functionality by depressing the lock button (e.g., the button depicting a locked lock in a darkened state) at the bottom of the speed button interface. If the user wishes to decrease the pump speed, the user must depress the speed down button (e.g., button with the “down” arrow) until the desired pump speed is achieved. In the third speed button interface of FIG. 6, the user has selected a pump speed of level 7 but has elected to keep the speed interface button unlocked by not depressing the speed lock button, thus the button depicting a locked lock is in a lightened state. In other instances, the unlocked state may be represented by depicting an unlocked lock. In the final speed button interface shown in FIG. 6, the user has locked the speed button interface by pressing the speed lock button, and the value ‘L’ is displayed as the user selects their speed. In other words, as the user toggles up and down through various speed increments, the value ‘L’ is displayed until the user stops pressing the speed buttons and settles on a suitable speed increment.

In some examples, the first display of the first user interface 233 on the console 228 may provide, present or display various functional status indicators 236 related to operation of the blood pump (e.g., which may be patient parameters and/or pump parameters). The first display 233 may have a preconfigured, fixed layout that facilitates a user controlling operation of the blood pump and at which set points for the blood pump may be adjusted. The first display of the first user interface 233 may be further connected to an alarm of the console, and be configured to relay several distinct alarm conditions to the user, patient and practitioners of the circulatory support system.

The first display 233 may present the various functional status indicators 236 one at a time, two at a time, three at a time, four at a time, five at a time, six at a time or more, for example. Further, the various functional status indicators 236 may be presented in an array of one functional status indicator, two functional status indicators, three functional status indicators, four functional status indicators, five functional status indicators, six functional status indicators or more. Functional status indicators 236 may include, but are not limited to: blood pump connection status, blood pump position status, device connection status, device position status, ventricular assist device position status, ventricular assist device connection status, motor connection status, motor rotation status, motor operation status, blood pump suction status, blood pump flow status, blood pump occlusion status, blood flow rate through the blood pump status, restricted blood flow rate through the blood pump status, blood pump flow rate status, restricted blood pump flow rate status, device flow rate status, restricted device flow rate status, device occlusion status, calibration status, recalibration status, pump calibration status, pump recalibration status, blood pump calibration status, blood pump recalibration status, device calibration status, device recalibration status, battery level status, low battery level status, critically low battery level status, Mean Arterial Pressure (MAP) status, low Mean Arterial Pressure status, critically low Mean Arterial Pressure status, ventricular pressure status, low ventricular pressure status, critically low ventricular pressure status, left ventricular pressure status, low left ventricular pressure status, critically low left ventricular pressure status, blood pump power status, device power status, first screen power status, second screen power status, alarm status, alarm on status, alarm off status, backlight status, backlight on status or backlight off status or any equivalent functional status indicator known in the art.

The first display 233 may present functional status indicators 236 as icons, bars, dots, symbols, indicia, or any other equivalent marking or designation known in the art. The aforementioned markings and designations may also be color-coded. Icons representing circulatory support system components and patient parameters may also be present and displayed on the first display 233. Such icons include, but are not limited to: a pump icon, a motor icon, a device icon, a device position icon, a pump position icon, a power status icon, a low power icon, a critically low power icon, a blood pump position icon, a device fit icon, a pump fit icon, a blood pump fit icon, a device connectivity icon, a pump connectivity icon, a blood pump connectivity icon, a flow-rate icon, a Mean Arterial Pressure icon, a pulse rate icon, a blood pressure icon, a blood flow-rate icon, an occlusion icon, a pump occlusion icon, a device occlusion icon, a blood pump occlusion icon, a line occlusion icon, a conduit occlusion icon, a pump pressure icon, a pump suction icon, a blood pump pressure icon, a blood pump suction icon, a device pressure icon, a device suction icon, an impeller icon, a battery level icon, a low battery level icon, and a critically low battery level icon. In some instances, normal operation of the blood pump may be indicated by illumination of the functional status indicators 236 in a first color of light, such as white light. In some instances, normal operation of the blood pump may further be indicated by illumination of a bar below the relevant icon or indicia of the status indicator, such as illumination of a bar in white light. A continuous row of aligned bars (e.g., white illuminated bars) below the functional status indicators 236 may be indicative of normal operation of the blood pump.

Several alarm conditions presented by the first display 233 of the first user interface are contemplated. For example, a first-level, low-level or cautionary alarm or alert may be indicated by illumination of a bar with a second, different color, such as a yellow bar of light situated in the vicinity of an appropriate icon on the first display 233. For instance, if the motor condition of the blood pump is worse than ideal, a yellow bar is presented beneath the motor icon on the first display 233 of the first user interface. Further, an accompanying level-one or cautionary alarm may sound on the console 228, and this sound is paired with both the yellow indicator on the first display 233 and a corresponding colored light (e.g., flashing yellow light) displayed via the backlight 241 on the rear of the console 228. The illuminated (e.g., flashing, steady, etc.) backlight 241 serves as a tertiary alarm to alert all capable persons within the vicinity to take action regarding operation of the circulatory support system. Additionally, a level-two, high-level or critical alarm or alert may be indicated by illumination of a bar with a third, different color, such as a red bar of light situated in the vicinity of an appropriate icon on the first display 233. For instance, if the motor condition of the pump is critically poor, a red bar is presented beneath the motor icon on the first display 233 of the first user interface. Further, an accompanying level-two or critical alarm may sound on the console 228, and this sound is paired with both the red indicator on the first display 233 and a corresponding colored light (e.g., flashing red light) displayed via the backlight 241 on the rear of the console 228.

Additional alarm conditions, displays, sounds, and alerts are also contemplated. A level-one alarm may be accompanied by a first sound, while a level-two alarm of a heightened level or responsiveness, severity, or criticality may be accompanied by a second sound, the second sound being different than the first sound. Further, a level-one alarm condition may be accompanied by a first grouping of sounds, the grouping of sounds may be sequential, alternating, patterned, combined, or comprise a rotation of sounds selected from the grouping in which one sound is played for the initial first-level alarm alert but a different sound is incorporated for each successive first-level alarm alert, each different sound selected from the grouping of level-one alarm condition sounds. Additionally, a level-two alarm condition may be accompanied by a second grouping of sounds, the second grouping of sounds being different than the first grouping of sounds. The second grouping of sounds may be sequential, alternating, patterned, combined, or comprise a rotation of sounds selected from the second grouping in which one sound is played for the initial second-level alarm alert but a different sound is incorporated for each successive second-level alarm alert, each different sound selected from the grouping of level-two alarm condition sounds.

The console 228 of the device may also coordinate the action of a second-level alarm or critical alarm alert. In this second-level alarm or critical alarm condition, an abnormal status indicator (e.g., red status bar) may appear near the appropriate icon on the first display 233 of the first user interface. For example, if the patient's Mean Arterial Pressure is too far beneath a predetermined threshold programmed into the console 228, then a second-level alarm is instantly issued, displaying a red status bar immediately above the Mean Arterial Pressure icon on the first display 233 of the first user interface. Simultaneously, the backlight 241 of the console 228 may illuminate a colored light (e.g., a flashing red light) corresponding to the second-level alarm condition to indicate the issuance of a second-level or critical alarm. Further, concomitantly with the two visual alarms, a second-level or critical alarm alert may be sounded from the console 228, alerting all capable persons within aural vicinity to the second-level or critical alarm condition of the circulatory support system.

The positioning of alarm indicators may be anywhere on the console 228 of the device to suit design considerations. In certain instances, the highest-level alarm (i.e., second-level alarm) is presented at the top of all status indicators 236. In other instances, the highest-level alarm is presented at the bottom of all status indicators or sandwiched between other available status indicators. Accordingly, the first-level alarm or cautionary alarm may be positioned anywhere along the console 228 as is contemplated for all other alarm conditions, levels and scenarios.

The console may further include various tactile or haptic feedback buttons and/or features. For example, and as shown in FIG. 6, a series of speed control buttons 240 may be present on the front of the console 228 or any other desirable area of the console 228. Other tactile and/or haptic feedback buttons and/or features include, but are not limited to: lock buttons or features, unlock buttons or features, speed up buttons or features, speed down buttons or features, speed lock buttons or features, speed unlock buttons or features, backlight on buttons or features, backlight off buttons or features, backlight luminosity buttons or features, backlight display buttons or features, backlight color buttons or features, pump on buttons or features, pump off buttons or features, device on buttons or features, device off buttons or features, volume level buttons or features, volume up buttons or features, volume down buttons or features, volume lock buttons or features, volume unlock buttons or features, mute buttons or features, unmute buttons or features, motor on buttons or features, motor off buttons or features, battery saver buttons or features, battery saver off buttons or features, battery saver on buttons or features, wireless connection buttons or features, wired connection buttons or features, pump speed buttons or features, increase pump speed buttons or features, decrease pump speed buttons or features, impeller buttons or features, increase impeller speed buttons or features, decrease impeller speed buttons or features, pump suction buttons or features, increase pump suction buttons or features, decrease pump suction buttons or features, motor rotation buttons or features, decrease speed of motor rotation buttons or features, increase speed of motor rotation buttons or features or any equivalent buttons or features known in the art.

The second display 235 of the second user interface is configured to display various real-time patient parameters or values derived from measured and/or estimated data obtained from the blood pump of the circulatory support system. The various real-time patient parameters or values may be presented or displayed one at a time, two at a time, three at a time, four at a time, five at a time, six at a time, seven at a time, eight at a time, nine at a time or more. Unless stated to the contrary, the terms “value” and “parameter” are synonymous within the context of the functionality of the second display.

The various real-time patient parameters may also be presented or displayed in an array of one real-time patient parameter, two real-time patient parameters, three real-time patient parameters, four real-time patient parameters, five real-time patient parameters, six real-time patient parameters, seven real-time patient parameters, eight real-time patient parameters, nine real-time patient parameters or more. The various real-time patient parameters may be presented in a grid, a patterned array, an irregular array, a horizontal array, a vertical array, a diagonal array, a circular array, an elliptical array, a rectangular array, a square array, a trapezoidal array or any known or equivalent display pattern known in the art.

Real-time patient parameters may include, but are not limited to: minimum arterial pressure, maximum arterial pressure, Mean Arterial Pressure (MAP), Mean Arterial Pressure waveform (MAP waveform), left ventricular pressure, left ventricular pressure waveform, ventricular pressure, blood flow rate, minimum blood flow rate, maximum blood flow rate, mean blood flow rate, blood flow rate through a blood flow pump of a circulatory support device, minimum blood flow rate through a blood flow pump of a circulatory support device, maximum blood flow rate through a blood flow pump of a circulatory support device, mean blood flow rate through a blood flow pump of a circulatory support device, motor operation status, pump operation status, blood pump operation status, device operation status, battery level status, low battery level status, critically low battery level status, device power status, pump power status, blood pump power status, pump position status, blood pump position status, device position status, pump occlusion status, blood pump occlusion status, device occlusion status, flow rate restriction status, pump flow restriction status, blood pump flow restriction status, device flow restriction status, alarm status, alarm on status, alarm off status, backlight status, backlight on status or backlight off status or any equivalent parameter known in the art or appropriate for the intended use of the present disclosure.

The second display 235 may present various real-time patient parameters or values derived from measured and/or estimated data obtained from the blood pump of a circulatory support system. For instance, real-time patient parameters or values representing measured data obtained from the blood pump of the circulatory support system may be shown in solid-line font or format, whereas real-time patient parameters or values derived from estimated data may be shown in a dashed-line, dotted-line or cross-hatched font or format. Values and parameters displayed in waveform are also subject to the same criteria, i.e., if the values and parameters displayed in waveform represent measured data, those values and parameters may be displayed in solid-line, whereas, those values and parameters derived from estimated data may be displayed in a dashed-line, dotted-line or cross-hatched waveform format.

For example, if the Mean Arterial Pressure is measured directly from a pressure sensor attached to the blood pump and positioned in the aorta of the patient, the Mean Arterial Pressure waveform 244 presented on the second display 235 may be shown in a sold-line waveform format. Additionally, the Mean Arterial Pressure 260 displayed as a number (i.e., 46 mm/Hg) may also be displayed in a solid-line font simultaneously with the presentation of the Mean Arterial Pressure waveform 244 being shown in a solid-line waveform format.

Additionally or alternatively, if any or all various real-time patient parameters or values fail to meet a predetermined threshold programmed into one or more of the console, circulatory support system, first user interface, first display, second user interface or second display, such real-time patient parameters or values may be shown in a dashed-line, dotted-line or cross-hatched font or format. Values and parameters displayed in waveform are also subject to the same criteria, i.e., if the values and parameters displayed in waveform fail to meet a predetermined threshold, those values and parameters may be displayed in a dashed-line, dotted-line or cross-hatched waveform format.

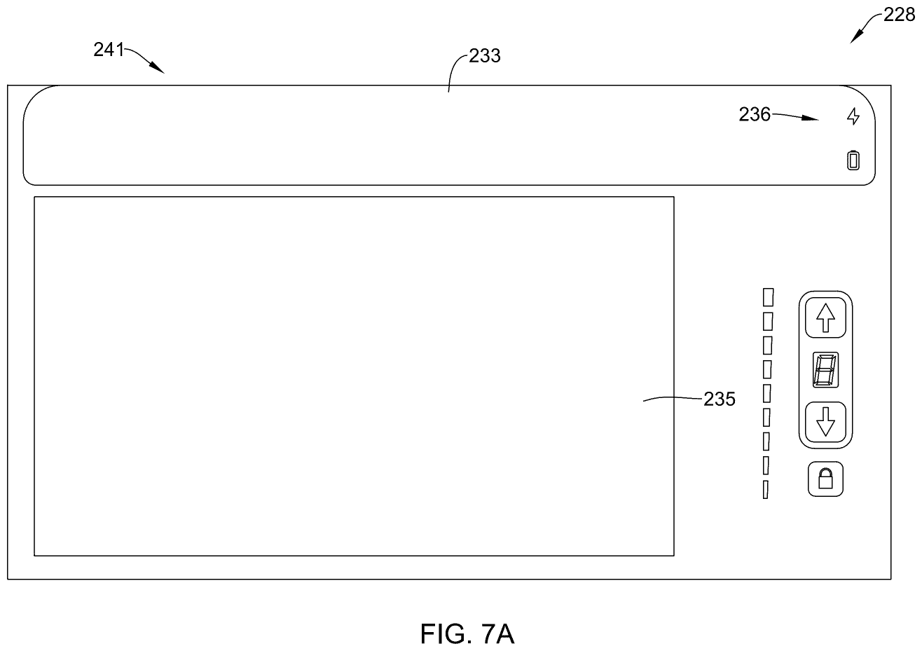

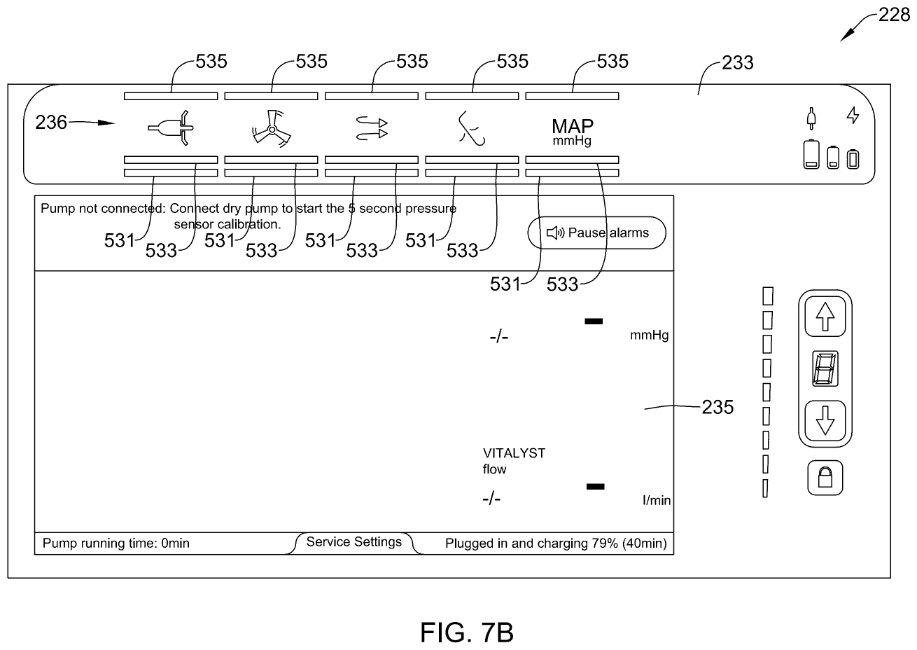

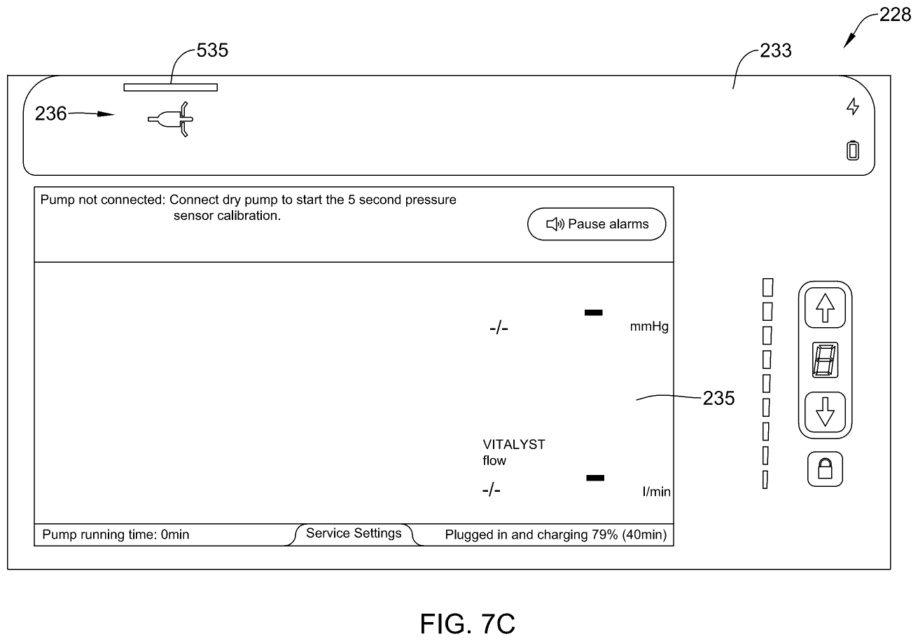

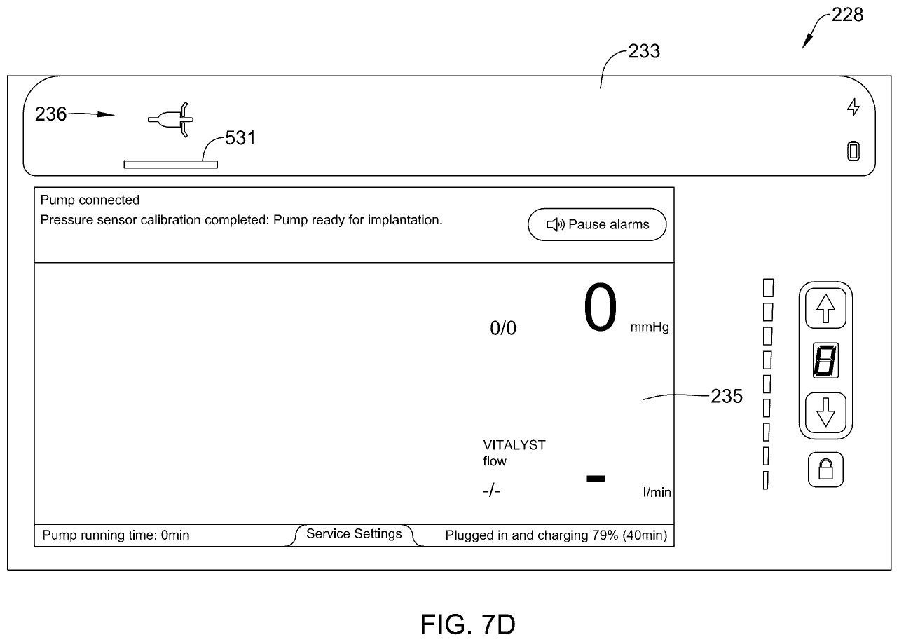

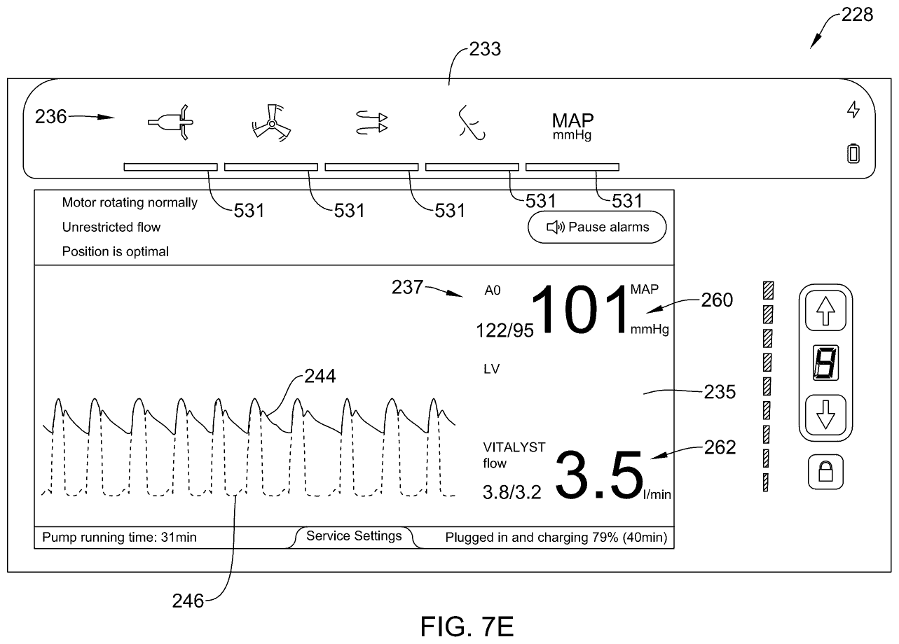

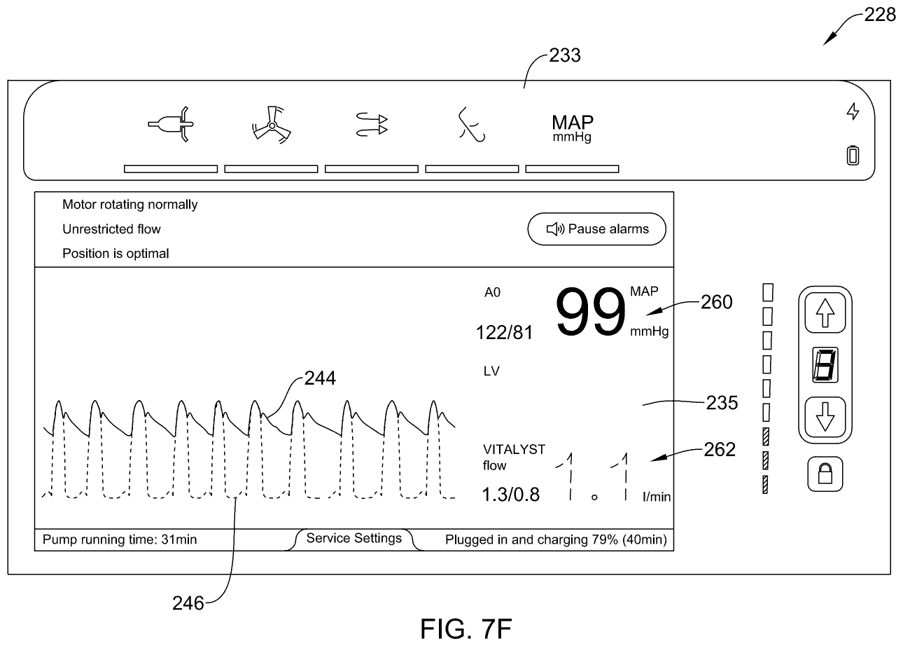

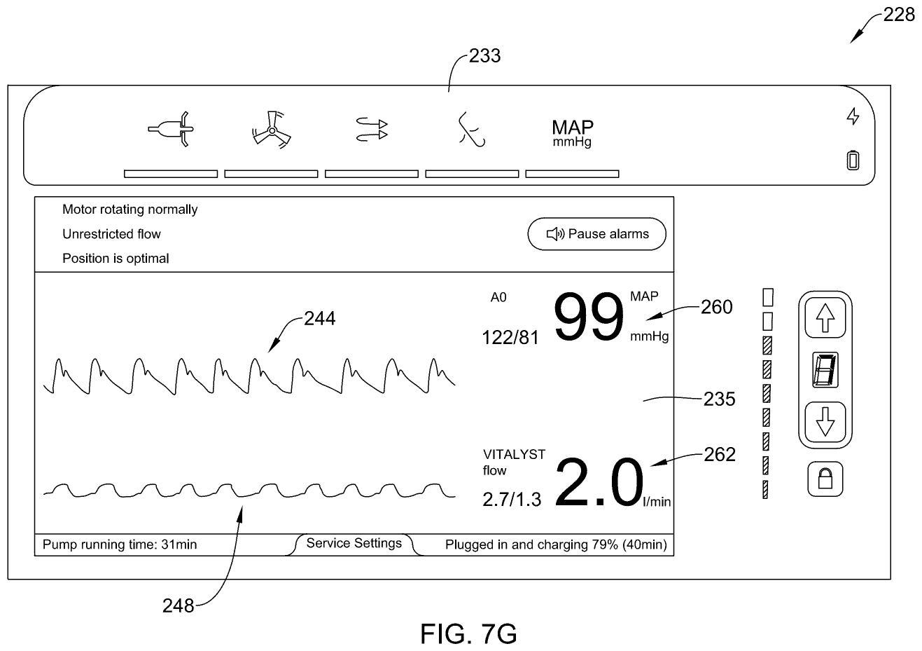

For example, if the left-ventricular pressure is recorded and/or calculated at a value less than a predetermined threshold, the left-ventricular pressure waveform 246 presented on the second display 235 may be shown in a dashed-line, dotted-line or cross-hatched waveform format. Additionally, the ventricular pressure displayed as a number may also be displayed in a dashed-line, dotted-line or cross-hatched font simultaneously with the presentation of the left-ventricular pressure waveform 246 being shown in dashed-line, dotted-line or cross-hatched waveform format. Additionally or alternatively, if the blood flow rate through the blood pump is calculated at a value less than a predetermined threshold, the blood flow rate waveform 248 (see FIG. 7G) presented on the second display 235 may be shown in a dashed-line, dotted-line or cross-hatched waveform format. Additionally, the blood flow rate displayed as a number may also be displayed in a dashed-line, dotted-line or cross-hatched font simultaneously with the presentation of the blood flow rate waveform 248 being shown in dashed-line, dotted-line or cross-hatched waveform format.

In some instances, if the system determines that any or all various real-time patient parameters or values shown on the second display 235 have a low confidence of accuracy and/or its accuracy is deemed uncertain, such real-time patient parameters or values may be shown in a dashed-line, dotted-line or cross-hatched font or format. Values and parameters displayed in waveform are also subject to the same criteria, i.e., if the values and parameters displayed in waveform have a low confidence of accuracy and/or its accuracy is deemed uncertain, those values and parameters may be displayed in a dashed-line, dotted-line or cross-hatched waveform format.

For example, if the blood flow rate through the blood pump is too low to provide an accurate estimate, the blood flow rate waveform 248 (see FIG. 7G) presented on the second display 235 may be shown in a dashed-line, dotted-line or cross-hatched waveform format. Additionally, the blood flow rate displayed as a number may also be displayed in a dashed-line, dotted-line or cross-hatched font simultaneously with the presentation of the blood flow rate waveform 248 being shown in dashed-line, dotted-line or cross-hatched waveform format.

The second display 235 may be configured to display screen layouts customizable and/or adjustable by users or practitioners (e.g., by medical professionals and/or other users of the second display), to provide data for multiple pumps and/or multiple patients simultaneously.

FIG. 7A shows an image of the console 228 where the backlight 241 is off and the first display 233 and the second display 235 are blank except for the presence of functional status indicators 236 in the right-side of the first display. The functional status indicator present include device power status (shown as an illuminated lightning bolt) and battery level status (shown as an illuminated battery). When in normal operation, these functional status indicators may be illuminated in a first color (e.g., white light). However, when a first-level alarm condition is present, the color of the status indicators may turn to a different color (e.g., yellow) and/or the status indicator may begin flashing, for example. In some instances, in the event of a second-level alarm condition, the color of the status indicators may turn to a third, different color (e.g., red) and/or the status indicator may begin flashing or flash at a different frequency/duration, for example. For example, when the battery has sufficient electrical charge, the battery indicator may be illuminated with a first color (e.g., white light), when the battery level is drained to a threshold level the battery indictor may change to a second color (e.g., yellow), and when the battery level is critically low, the battery indicator may be illuminated with a third color (e.g., red light) and/or begin flashing.

FIG. 7B shows an image where most functional status indicators 236 of the first display 233 are presented. Bars and indicia 531 of a first color (e.g., white) indicate proper functioning by the status indicators relating to the operation of a blood pump of a circulatory support system. Bars and indicia 533 of a second color (e.g., yellow) indicate a cautionary or first-level alarm condition concerning the functional status indicators relating to the operation of a blood pump of a circulatory support system. Bars and indicia 535 of a third color (e.g., red) indicate a critical or second-level alarm condition concerning the functional status indicators 236 relating to the operation of the blood pump of the circulatory support system. Alternative color-codes and indicia displays are contemplated for all alarm levels and all embodiments of the present disclosure.

FIG. 7C shows a red-bar 535 of a functional status indicator 236 indicating that the blood pump is not connected with the circulatory support system. This indicator may be depicted in red to communicate a critical or second-level alarm condition concerning the operation of the blood pump of a circulatory support system. A similar red-bar 535 may be illuminated above any one of the other functional status indicators 236, indicating a critical or second-level alarm condition is present for the respective aspect of the blood pump.

FIG. 7D shows a white-bar 531 of a functional status indicator 236 communicating that the blood pump of the circulatory support system is properly placed and in the correct positioning. A similar white-bar 531 may be illuminated below any one of the other functional status indicators 236, indicating normal operation for the respective aspect of the blood pump. A plurality of white-bars 531 aligned across the bottom of the first display 233 may be indicative that the entire circulatory support system is running normally.

FIG. 7E shows a full functioning display for both the first display 233 and the second display 235 of an exemplary console 228 of the present disclosure. As shown, all functional status indicator bars are illuminated white, depicting that all functional status indicators 236 displayed are communicating proper function of the circulatory support system. Further presented on the second display 235 is a waveform 244 depicting Mean Arterial Pressure measured continuously in real-time from a pressure sensor of the blood pump of the circulatory support system positioned in the aorta of a patient. Also shown numerically are the patient parameters 260 of Mean Arterial Pressure value (in larger font), along with minimum arterial pressure value (in smaller font) and maximum arterial pressure value (in smaller font) associated therewith. These patient parameters of Mean Arterial Pressure (as well as minimum and maximum arterial pressure values) may be updated on the display in real time (e.g., with every heartbeat of the patient). A waveform 246 depicting mean ventricular pressure estimated continuously in real-time from an algorithm of the circulatory support system is also depicted in FIG. 7E.

Also shown in FIG. 7E is the patient parameters 262 of blood flow rate through the blood pump, with the mean blood flow rate through the blood pump shown in larger font, along with minimum and maximum blood flow rates through the blood pump shown in smaller font. The values may be updated in real-time, e.g., for each beating of the patient's heart.

FIG. 7F shows a similar display presentation to FIG. 7E, except the blood flow rate through the pump has failed to meet a predetermined threshold and/or the system has determined that the displayed value has low confidence of accuracy and/or its accuracy is uncertain, therefore the blood flow rate values are shown in cross-hatched font. In FIG. 7F, the mean blood flow rate is shown in a larger font than both the minimum and maximum blood flow rate, and all blood flow rate values are displayed in cross-hatched font.

FIG. 7G shows another depiction of information displayed on the second display 235, including a real-time waveform 248 of the mean blood flow rate through the blood pump over a period of time. In an instance in which the system has determined that the values for the displayed waveform 248 have low confidence of accuracy and/or its accuracy is uncertain, the waveform 248 may be shown in cross-hatched font.

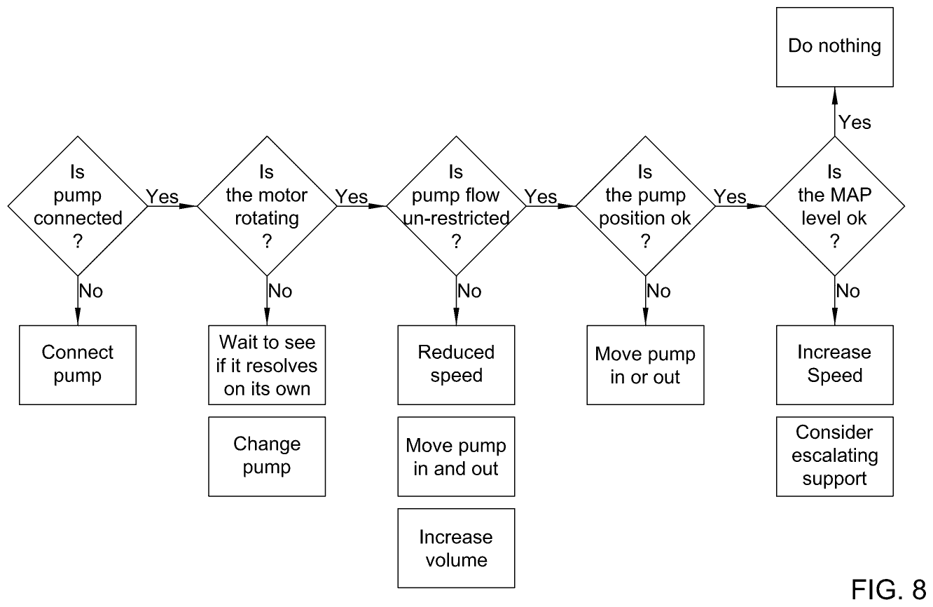

FIG. 8 depicts a flow chart that relates to contemplated methods of the present disclosure. The flow chart depicts several operating conditions of a circulatory support system and shows a troubleshooting guide with programmed instructions that are to be displayed on one or both of the first display 233 and the second display 235 in the event these conditions are to arise.

As discussed, the console 228 may be configured to communicate with the blood pump and/or the first and second user interface(s) via a wired and/or wireless connection. In some examples, the console 228 may be configured to communicate with the blood pump via a wired connection, communicate with a first and/or second user interface via a transmission control protocol (TCP) connection or a RS232 protocol connection, and communicate with a first or second user interface via a TCP connection or a RS232 protocol connection. Additionally or alternatively, the console 228 may be configured to communicate with the blood pump via a wired connection, communicate with a first or second user interface via a TCP connection or RS232 connection, and communicate with a first or second user interface via a TCP connection or WiFi connection. Other suitable configurations are contemplated.

As discussed, the first and/or second user interface(s) 233/235 may be configured to communicate with the console(s) 228 over one or more networks. For example, the first and/or second user interface(s) 233/235 may be configured to communicate with the console(s) 228 and/or a server (e.g., a remote server or local server) over one or both of a local area network (LAN) and/or a wide area network (WAN) to receive data (e.g., live data, historical data, etc.) from the console(s) 228 and/or one or more servers. The first and/or second user interface(s) 233/235 may communicate with one or more consoles over a LAN and the first and/or second user interface(s) 233/235 may communicate with one or more consoles over a WAN, but other suitable network connections are contemplated. In some cases, the LAN may be a network local to a building or company system and may include wired and/or wireless network connections between computing devices of the network. The WAN may be a network that may have or include a cloud computing connection (e.g., a web server connection) and/or other suitable connection for connecting with devices that may or may be part of the LAN. The WAN may include wired and/or wireless network connections. When a web server or other server is utilized, the server may be configured to translate data (e.g., a data stream) into a format (e.g., a web page, a web socket, app data, etc.) that facilitates allowing users to consume the data via the first and/or second display(s) 233/235.

As discussed, the circulatory support system may be utilized in one or more illustrative techniques for displaying information and/or data related to operation of the blood pump to users. In some examples, the console 228 of the circulatory support system may receive information and/or data over time from the blood pump. The information and/or data received over time may be related to the patient, the blood pump, and/or operation of the blood pump (e.g., operation of the blood pump within the patient). As the information and/or data is received from the blood pump, the information and/or data received over time may be displayed live on the second user interface 235 (e.g., on the console display) of the console 228. In some examples, the information and/or data received over time may be displayed in a preconfigured, fixed layout or configuration to a facilitate a portable nature of the console 228 and effectively relaying patient and/or blood pump related information to users of the console 228.

Methods of operation of a circulatory support system of the present disclosure include but are not limited to: utilizing a user display of a user interface to present real-time patient parameter values based on the measured and/or estimated real-time data obtained from a blood pump of a circulatory support device such as a ventricular assist device (VAD). Contemplated methods further include receiving instructions generated by the user display during a first-level alarm condition or a second-level alarm condition, whereby the user display sounds and displays an alarm connected with a backlight 241 of the console 228 of the circulatory support system to alert a user of the circulatory support system to a first-level or second-level alarm condition of the VAD. The alarm is configured to provide one or more audio and/or visual alerts provided by the user interface that simultaneously correspond with a visual alert provided by the backlight 241 of the console 228 of the circulatory support system.

During an alarm condition or alarm scenario, methods of the present disclosure further contemplate a first-level alarm condition that is accompanied by a light of a first color displayed simultaneously on both the user display 233/235 and the backlight 241; and a second-level alarm condition that is accompanied by a light of a second color displayed simultaneously on both the user display 233/235 and the backlight 241. The second color being different than the first color. Further, the first-level alarm condition is accompanied by a first sound, and the second-level alarm condition is accompanied by a second sound, the first sound being different than the second sound.

Additionally, during an alarm condition or scenario, methods of the present disclosure further contemplate that the alarm provides audio feedback regarding one or more first- and/or second-level alarm conditions. The one or more first- and/or second-level alarm conditions may be selected from a group of functional status indicators comprising: low battery status, critically low battery status, improper ventricular assist device connection, improper ventricular assist device position, improper pump connection, improper pump position, motor status, motor rotation status, motor speed status, blood flow rate status, blood flow rate though the blood pump status, restricted flow rate status, flow occlusion status, ventricular assist device occlusion status, pump occlusion status, calibration status, recalibration status, Mean Arterial Pressure (MAP) status, alarm status, lock status, unlock status, backlight status, pump speed status, pump operational status, ventricular assist device status or any equivalent functional status indicators mentioned elsewhere in this disclosure or known in the art.

Multiple user interfaces, user displays, and myriad arrays and sequences of alarms, alerts, presented data, presented values, indicators, blood pump devices, peripheral devices, medical components and sounds are also contemplated for incorporation into the methods of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A system for obtaining measured and/or estimated real-time data regarding patient use and function of a ventricular assist device (VAD), the system comprising:
a first user interface with a first display connected to a blood pump of the VAD and forming part of a console, the first display configured to present functional status indicators of the system, and
a second user interface integrated with the console, the second user interface further comprising:
a second display configured to present corresponding real-time patient parameter values based on measured and/or estimated real-time data obtained from a blood pump of the VAD;
wherein the console further comprises an alarm connected with a backlight of the console, wherein the alarm is configured to provide one or more audio and/or visual alerts provided by the first user interface that simultaneously correspond with a visual alert provided by the backlight.

2. The system of claim 1, wherein the backlight is positioned on a rear side of the console and the first display is positioned on a front side of the console.

3. The system of claim 1, wherein the alarm is configured to display a first-level alarm light of a first color via both the backlight and at a second position remote from the backlight on the first display simultaneously.

4. The system of claim 3, wherein the alarm is configured to display a second-level alarm light of a second color via both the backlight and at a second position remote from the backlight on the first display simultaneously, wherein the second color is different than the first color.

5. The system of claim 1, wherein the alarm is configured to display one of a second-level alarm light or a first-level alarm light based upon the measured and/or estimated real-time data failing to meet a predetermined threshold value for one or more parameters selected from the group comprising: Mean Arterial Pressure (MAP), blood flow rate through the blood pump, ventricular assist device connection status, pump connection status, ventricular assist device position status, pump position status, motor status, motor rotation status, motor speed status, flow occlusion status, flow restriction status, flow speed status, flow rate status or battery level status.

6. The system of claim 1, wherein the measured and/or estimated real-time data comprise one or more values selected from the group of: Mean Arterial Pressure (MAP), minimum arterial pressure, maximum arterial pressure, MAP waveform, estimated blood pressure, estimated blood pressure waveform, blood flow rate through the ventricular assist device, blood flow rate through a pump of the system, maximum blood flow rate through the ventricular assist device, maximum blood flow rate through a pump of the system, minimum blood flow rate through the ventricular assist device, minimum blood flow rate through a pump of the system or left ventricular pressure.

7. The system of claim 1, wherein the functional status indicators of the system presented by the first display comprise one or more functional status indicators selected from the group of: low battery status, critically low battery status, ventricular assist device connection, ventricular assist device position, pump connection, pump position, motor status, motor rotation status, motor speed status, flow rate status, restricted flow rate status, flow occlusion status, ventricular assist device occlusion status, pump occlusion status, calibration status, recalibration status, Mean Arterial Pressure (MAP) status, alarm status, lock status, unlock status, backlight status, pump speed status, pump operational status or ventricular assist device status.

8. The system of claim 1, wherein when the estimated real-time data fails to meet a predetermined threshold, the second display presents the corresponding real-time patient parameter values in a cross-hatched or dashed-line font.

9. The system of claim 1, wherein the first display continues to present functional status indicators of the system in an event where the second user interface and/or second display becomes inoperable.

10. The system of claim 1, wherein when the minimum, maximum, or mean blood flow rate of the blood pump of the VAD fail to meet a predetermined blood flow rate threshold, each blood flow rate value failing to meet the predetermined blood flow rate threshold is presented on the second display in a cross-hatched or dashed-line font.

11. The system of claim 1, wherein the first user interface runs on a first operating system and the second user interface runs on a second operating system; the second operating system being a different operating system than the first operating system.

12. A system for obtaining measured and/or estimated real-time data regarding patient use and function of a ventricular assist device (VAD), the system comprising:
a first user interface connected to a blood pump of the VAD, the first user interface comprising a first display and forming part of a console, the first display configured to present functional status indicators of the system, and
a second user interface integrated with the console; wherein the second user interface comprises a second display and is configured to present corresponding real-time patient parameter values based on the measured and/or estimated real-time data obtained from a blood pump of the VAD;
wherein the system is configured to continue to operate the blood pump and the first display continues to present functional status indicators of the system in an event where the second user interface and/or second display becomes inoperable.

13. The system of claim 12, wherein the system further comprises an alarm integrated with the console.

14. The system of claim 12, wherein the first user interface runs on a first operating system and the second user interface runs on a second operating system; the second operating system being a different operating system than the first operating system.

15. The system of claim 12, wherein when the estimated real-time data fails to meet a predetermined threshold value, the second display presents the corresponding real-time patient parameter values in a cross-hatched or dashed-line font.

16. A system for obtaining measured and/or estimated real-time data regarding patient use and function of a ventricular assist device (VAD), the system comprising:
a first user interface with a first display connected to a blood pump of a VAD and forming part of a console, the first display configured to present functional status indicators of the system, and
a second user interface integrated with the console, the second user interface further comprising:
a second display configured to present real-time patient parameter values based on the measured and/or estimated real-time data obtained from a blood pump of the VAD; the real-time patient parameter values comprising Mean Arterial Pressure (MAP), minimum arterial pressure, maximum arterial pressure, and MAP waveform.

17. The system of claim 16, wherein the second display presents Mean Arterial Pressure (MAP) in a larger font than what is presented for minimum and maximum arterial pressure.

18. The system of claim 16, wherein the second display presents MAP waveform in full-line format for values that meet a predetermined threshold, and in dashed-line format for values that fail to meet a predetermined threshold.

19. The system of claim 16, wherein the minimum arterial pressure value, maximum arterial pressure value, and mean arterial pressure values are updated on the second display with each patient heartbeat.

20. The system of claim 16, wherein the second display further presents:
a.) a mean blood flow rate through the blood pump of the ventricular assist device;
b.) a maximum blood flow rate through the blood pump of the ventricular assist device;
c.) a minimum blood flow rate through the blood pump of the ventricular assist device; and
wherein the font size of the presented mean blood flow rate is larger than the font size of the presented minimum and maximum blood flow rates.
